SEQUENCE LISTING (21 sequences)

SEQ ID NO: 1 - DNA, 1250 bp, Zea mays
gaacctcgccggcggccgcagccgcatagaccggaagagaaaggaagcagagatcggaggcaggcgaagatggggcgcggcaaggtgcagctgaagcggatagagaacaagataaaccggcaggtgaccttctccaagcgccggaacgggctgctgaagaaggcgcacgagatctccgtcctctgcgacgccgaggtcgccgtcatcgtcttctcccccaagggcaagctctacgagtacgcctccgactcccgcatggacaaaattctagaacgttatgagcgatattcctatgctgaaaaggctcttatttcagctgaatctgaaagtgagggaaattggtgccacgaatacaggaaactgaaggccaaaattgagaccatacaaagatgccacaagcacctgatgggagaggatctagagtctttgaatccaaaagagctccaacaactagagcagcagctggagagctcactgaagcacatcagatcaagaaagagccaccttatggccgagtcaatttctgagctacagaagaaggagaggtcactgcaggaggagaacaagattctacagaaggaactttcagagaggcagaaggcggtcgctagccggcagcagcagcagcagcaggtgcagtgggaccagcagacacaggtccaggtccagacaagctcatcgtcttcttccttcatgatgaggcaggatcagcagggactgccacctccacaaaacatctgcttcccgccgttgagcatcggagagagaggcgaagaggtggctgcggcggcgcagcagcagctgcctcctccggggcaggcgcaaccacagctccgcatcgcaggtctgccgccatggatgctgagccacctcaatgcataaggagggcgagcaaatggcgtgcgaagtgattgattgctcaccgttgattgaacgctagcctaagttcatggcggcagcaaactaagctaaaactattgttatgtttgcaaggaagggtaacccgctgtgtaatctttgtccagctagcatgtaccaactgagatgcatgcatgcttattattgtccaattacccgtgaatctagcggtgcttttggtgagaggccggccggacgggtgcagtttacttcaaatatggtttgtgattttgtgtaaatagtattaactaggagtcctatggtaagtaaattaattaatgggaggatgcatgaataaaacctctcttgtgctgcaaaaaaaaaaaaaaaaa

SEQ ID NO: 2 - protein, 270 aa, Zea mays
MGRGKVQLKRIENKINRQVTFSKRRNGLLKKAHEISVLCDAEVAVIVFSPKGKLYEYASDSRMDKILERYERYSYAEKALISAESESEGNWCHEYRKLKAKIETIQRCHKHLMGEDLESLNPKELQQLEQQLESSLKHIRSRKSHLMAESISELQKKERSLQEENKILQKELSERQKAVASRQQQQQQVQWDQQTQVQVQTSSSSSSFMMRQDQQGLPPPQNICFPPLSIGERGEEVAAAAQQQLPPPGQAQPQLRIAGLPPWMLSHLNA

SEQ ID NO: 3 - DNA, 20 bp, Zea mays
atggggaggggaaggattga

SEQ ID NO: 4 - DNA, 21 bp, Zea mays
ctgttgttggcgtactcgtag

SEQ ID NO: 5 - DNA, 21 bp, Zea mays
aggggcaagatcgacatcaag

SEQ ID NO: 6 - DNA, 19 bp, Zea mays
gkcgwactcgtagaggcgg

SEQ ID NO: 7 - DNA, 20 bp, Zea mays
atggggaggggacgmgttga

SEQ ID NO: 8 - DNA, 20 bp, Zea mays
gctgccgaactcgtagagct

SEQ ID NO: 9 - DNA, 21 bp, Zea mays
gttgttggcgtactcgtagag

SEQ ID NO: 10 - DNA, 20 bp, Zea mays
gggcgcaaggtacagctgaa

SEQ ID NO: 11 - DNA, 21 bp, Zea mays
gttgttggcgtactcgtagag

SEQ ID NO: 12 - DNA, 21 bp, Zea mays
aagggcaagactgagatcaag

SEQ ID NO: 13 - DNA, 23 bp, Zea mays
cacttgaactcttttacgcttat

SEQ ID NO: 14 - DNA, 23 bp, Zea mays
gacaatcttgacacatgtatgaa

SEQ ID NO: 15 - DNA, 20 bp, Zea mays
ctcatcgttgattgaacgct

SEQ ID NO: 16 - DNA, 21 bp, Zea mays
agaggttttattcatgcatcc

SEQ ID NO: 17 - DNA, 29 bp, Zea mays
actggtaccagcaaatggcgtgcgaagtg

SEQ ID NO: 18 - DNA, 29 bp, Zea mays
cgcaagcttgcagcacaagagaggtttta

SEQ ID NO: 19 - DNA, 21 bp, Zea mays
ctgaagcacatcagatcaaga

SEQ ID NO: 20 - DNA, 20 bp, Zea mays
actcatgaggtagtgacatt

SEQ ID NO: 21 - DNA, 20 bp, Zea mays
catttagcaggtcactgtac

(19) United States
(12) Patent Application Publication
(10) Pub. No.: US 2003/0018995 A1
(43) Pub. Date: Jan. 23, 2003

(54) Plants with a modified flower and seed development

(75) Inventors: Thomas Dresselhaus, Hamburg (DE); Sigrid Heuer, Hamburg (DE); Horst Lorz, Hamburg (DE)

Correspondence Address: Norris, McLaughlin & Marcus P.A., 30th Floor, 220 East 42nd Street, New York, NY, 10017, US

(73) Assignee: Westdeutsche Saatzucht H.R. Path, Rastatt (DE)

(21) Appl. No.: 10/105,021

(22) Filed: Mar. 22, 2002

Related U.S. Application Data
(63) Continuation of application No. PCT/EP00/10484, filed on Oct. 25, 2000.

(30) Foreign Application Priority Data
Oct. 25, 1999 (EP) 99120842.2

(51) Int. Cl. A01H 5/00; C07H 21/04; C12N 9/00; C12N 5/04

(52) U.S. Cl. 800/287; 800/320.1; 435/320.1; 435/419; 435/183; 536/23.6

(57) ABSTRACT

The present invention relates to nucleic acid molecules enabling the specific production of a plant exhibiting a modified flower development and/or autonomous embryo and/or endosperm development as components for manipulating apomixis. The invention also relates to methods to obtain said transgenic plants and to methods for isolating flower specific genes.

### CROSS-REFERENCES TO RELATED APPLICATIONS

This application is a continuation to PCT/EP00/10484, which was filed on Oct. 25, 2000 and which claimed priority to EP application 99120842.2, which was filed on Oct. 25, 1999.

### SPECIFICATION

The present invention relates to isolated nucleic acid molecules useful for the production of plants with a modified flower and seed development in particular male and female sterility, in particular precocious embryo and/or endosperm development, in particular monocotyledonous plants, to vectors containing the nucleic acid molecules, to host cells containing the vectors, to plants, harvest and propagation material containing the host cells, to methods for obtaining them and to methods for isolating such nucleic acid molecules.

The introduction of genes into transgenic plants is considered to have high commercial value. The transfer of heterologous genes or genes of interest into a plant under control of tissue-specific regulatory elements provides a powerful means of conferring selective advantages to plants and to increase their commercial value. The ability to control gene expression is useful for conferring resistance and immunity to certain diseases or to modify the metabolism of a tissue. Plant genetic engineering techniques also prove useful in generating improved plants for plant breeding purposes, such as male sterile plants. Finally, plant genetic engineering might also be used for the production of plants exhibiting a modified development of their flowers and/or fruits. Such plants are of commercial interest as they might be able to form an increased number of fruits, fruits with an increased size, or fruits with a modified structure or function. Furthermore, the maturation period of fruits and flowers may be shortened or adapted to environmental factors. Male and female sterility are important traits for fast breeding and F1-hybrid seed production.

Mutations in various genes controlling transition from vegetative growth to flower formation and development were described during the last 10 years. The interactions of proteins involved in the regulation of flower organ formation/identity were summarised in the so called ABC-model (Coen and Meyerowitz, 1991) Many genes leading to homeotic transformation of flower organs once inactivated, or ectopically expressed, encode MADS domain transcription factor proteins. The functional role of most MADS domain proteins is linked to floral meristem and organ identity (Richmann and Meyerowitz, 1997). Some genes were thus used to manipulate flower structure. E.g. Mandel et al. (1992a) have shown that altering the expression of a single regulatory gene may result in predictable manipulation of the tobacco flower structure. Up to now nearly all transgenic approaches were performed with dicotyledonous plants (see e.g. Mandel et al., 1992a; Richmann and Meyerowitz, 1997; Kater et al., 1998). Transgenic approaches to modify flower organ structure with monocotyledonous plants were obviously not successfully performed. A few mutations were reported with knock-outs in flower regulatory genes of monocotyledonous species. Unfortunately, the expected change e.g. in maize sex organs once the C-function gene ZAG1 was mutated, did not affect the identity of reproductive organs (Mena et al., 1996). Recently, parthenogenetic fruit development was successfully engineered genetically (Rotino et al., 1997), but again only using dicotyledonous plant species.

Another biological process linked to flower and seed development is apomixis (asexual reproduction through seeds: Koltunow et al., 1995; Vielle-Calzada et al., 1996). Due to the enormous economical potential of apomixis once controllable in sexual crops, its application was named after the ‘Green Revolution’ as the ‘Asexual Revolution’ (Vielle-Calzada et al., 1996). Up to now all approaches to isolate the ‘apomixis genes’ from apomictic species failed. Genes involved in autonomous endosperm development once inactivated were recently isolated from Arabidopsis (see Ohad et al., 1999; Luo et al., 1999). Autonomous embryo development (via parthenogenesis) , a further component of apomixis will be necessary to engineer the apomixis trait in sexual crops. E.g. in wheat, lines have been described producing up to 90% parthenogenetic haploids (Matzk et al., 1995). Almost no molecular data concerning parthenogenesis is available for higher plants: one protein (α-tubulin) was identified from the above described wheat lines whose expression is associated with the initiation of parthenogenesis (Matzk et al., 1997). Nevertheless, such a ‘house keeping gene’ will not be a valuable tool for genetic engineering of the induction of parthenogenesis. Regulatory genes are needed.

Thus, it is considered particularly important to develop and provide means and methods that allow the production of plants exhibiting a modified flower, fruit and or seed development.

Thus, the technical problem underlying the present invention is to provide nucleic acid molecules for use in cloning and expressing genes involved in flower, seed and/or fruit development, in particular for use in monocotyledonous plants which allow the production of plants with a modified flower, seed and/or fruit development.

The present invention solves the technical problem underlying the present invention by providing purified nucleic acid molecules for use in cloning and expressing a flower specific or flower abundant gene in a plant encoding a protein influencing flower and/or fruit structure, function and/or development which are selected from the group consisting of

(a) the nucleic acid sequence defined in SEQ ID No. 1, or part or a complementary strand thereof,

(b) a nucleic acid sequence encoding a protein or peptide with the amino acid sequence defined in SEQ ID No. 2, or part or a complementary strand thereof,

(c) a nucleic acid sequence which hybridises to the nucleic acid sequence defined a) or b), or part or a complementary strand thereof and

(d) a nucleic acid sequence which is degenerate as a result of the genetic code to the nucleic acid sequence defined in a), b), c), or part or a complementary strand thereof,

(e) alleles or derivatives of the nucleic acid sequence defined in (a), (b), (c), (d), or part or a complementary strand thereof.

The nucleic acid sequence set out in SEQ ID No. 1 represents a nucleic acid sequence, namely a cDNA sequence encoding a protein, called the ZmMADS3 protein, which is essential for flower development and is active in flowers in particular in immature male and female flowers, but also in the mature embryo sac of maize. The ZmMADS3 protein is also active in nodes and adjacent cell layers, in particular of maize plants, i.e. that tissue from which the development of the female flower, namely the cob, initiates. This sequence will be termed in the following the coding sequence of the present invention or the ZmMADS3 coding sequence.

The amino acid sequence set out in SEQ ID No. 2 represents the amino acid sequence of the protein ZmMADS3.

The present invention also relates to nucleic acid sequences which hybridise, in particular under stringent conditions, to the sequence set out in SEQ ID No. 1. In particular, these sequences have a degree of identity of 70% to the sequence of SEQ ID No. 1.

In the context of the present invention, nucleic acid sequences which hybridise to the specifically disclosed sequence of SEQ. Id. No. 1 are sequences which have a degree of 60% to 70% sequence identity to the specifically disclosed sequence on nucleotide level. In an even more preferred embodiment of the present invention, sequences which are encompassed by the present invention are sequences which have a degree of identity of more than 70%, and even more preferred, more than 80%, 90%, 95% and particularly 99% to the specifically disclosed sequences on nucleotide level.

Thus, the present invention relates to nucleic acid sequences, in particular DNA sequences which hybridise under the hybridisation conditions as described in Sambrook et al., (1989) in particular under the following conditions to the sequences specifically disclosed:

Hybridisation buffer: 1 M NaCl; 1% SDS; 10% dextran sulphate; 100 μg/ml ssDNA

Hybridisation temperature: 65° C.

First wash: 2×SSC; 0.5% SDS at room temperature

Second wash: 0.2×SSC; 0.5% SDS at 65° C.

More preferably, the hybridisation conditions are chosen as identified above, except that a hybridisation temperature and second wash temperature of 68° C., and even more preferred, a hybridisation temperature and second wash temperature of 70° C. is applied.

Thus, the present invention also comprises nucleic acid sequences which are functionally equivalent to the sequence of SEQ ID No. 1, in particular sequences which have at least homology to the sequence of SEQ ID No. 1. The invention also relates to alleles and derivatives of the sequences mentioned above which are defined as sequences being essentially similar to the above sequences but comprising, for instance, nucleotide exchanges, substitutions (also by unusual nucleotides), rearrangements, mutations, deletions, insertions, additions or nucleotide modifications and are functionally equivalent to the sequence set out in SEQ ID No. 1.

The nucleic acid molecules of the present invention are, in a preferred embodiment, derived from maize (Zea mays).

According to the present invention it was found that the nucleic acid sequence isolated is specifically expressed in nodes and male and female flowers, in particular immature flowers and obviously plays an important role in flower, seed and fruit, in particular embryo and/or endosperm, development.

Thus, the nucleic acid molecules of the present invention are useful for cloning tissue specific, in particular seed, node and/or flower specific nucleic acid sequences, in particular regulatory elements, coding sequences and/or complete genes, in plants, in particular in monocotyledonous plants.

Thus, the present invention provides the means for the isolation of node, flower and embryo sac specific coding sequences and/or transcription regulatory elements that direct or contribute to node, flower and/or embryo sac preferred gene expression in plants, in particular in monocotyledonous plants, such as maize. The present invention also provides the means of isolating node, embryo sac and/or flower specifically expressed genes and their transcripts.

The nucleic acid molecules of the present invention are also useful for expressing or suppressing a node, embryo sac and flower specific protein, namely the ZmMADS3 protein and its target genes, in plants, in particular in the nodes, flower and/or embryo sac of plants such as maize or of dicotyledonous plants such as sugar beets (Beta vulgaris) Thus, the present invention provides the means to allow the expression or suppression of a particular node, embryo sac or flower specific or node, embryo sac or flower abundant gene in node or flower thereby enabling the modification of node, embryo sac, fruit or flower development, function and/or structure. In particular, the present invention may allow the production of plants, the embryos of which develop into plants without fertilisation and allow apomixis, i.e. the asexual production of seeds. The present invention enables the specific production of a plant exhibiting a modified flower development and an autonomous embryo and/or endosperm development as components for manipulating apomixis. The ZmMADS3 sequence of the present invention is in particular expressed in egg cells and zygotes after fertilisation. Further expression during later embryo development could not be detected. The ZmMADS3 protein coded by the nucleic acid sequence of the present invention may act as a repressor/activator of zygote/embryo development. Modification of the protein may lead to parthenogenetic embryo development, which is an important component of engineering the apomixis trait. The coding sequence of the present invention may be overexpressed in transformed plants due to expression under control of a strong constitutive, tissue or tissue specific or regulated promoter. It is also possible to modify the coding sequence of the present invention so as to allow the production of a modified node, embryo sac or flower specific protein, which in turn modifies in a desired manner node, embryo sac or flower development and/or function. Most importantly, the present invention provides the means to specifically inhibit the formation of a protein essential for node, embryo sac, flower and or fruit function or development, namely the ZmMADS3 protein, by transforming plants with antisense constructs comprising all or part of the coding sequence or, transcribed but not translated regions of the ZmMADS3 gene (UTR, untranslated region) or a part thereof in antisense orientation under the control of its wild-type or appropriate other regulatory elements so as to effectively bind to wild-type ZmMADS3 mRNA and inhibit its translation. Such a construct leads upon expression to the abolishment of the wild-type ZmMADS3 function thereby producing modified plants, for instance with an increased number of fruits or precocious embryo/endosperm development as components of engineering the apomixis trait in sexual crops.

Of course such an eliminating effect of natural gene function may also be obtained using co-suppression technology. Accordingly, the nucleic acid sequence of the present invention cloned in sense orientation to at least one regulating element, such as a promoter, into a suitable vector is transformed into a plant cell which in turn may exhibit a suppressed gene function of a wild-type ZmMADS3 gene.

The present invention also provides access to regulatory elements, such as promoters and 3′ transcription termination signals providing for flower, embryo sac or node specific expression of any gene of interest, including the ZmMADS3 coding sequence of the present invention. Such regulatory elements may be obtained by using the nucleic acid sequence of the present invention to isolate in a genomic DNA library hybridising sequences also encompassing regulatory elements located adjacent to the ZmMADS3 coding sequence.

In a particularly preferred embodiment of the present invention, the above defined promoter of the present invention is expressed in a spatially and temporarily specific manner, preferably in immature male or female flowers, embryo sacs or nodes. In a further preferred embodiment the promoter of the present invention is able, due to specific sequence elements present in its sequence, to direct expression in the above mentioned tissues. Accordingly, the proteins encoded by the gene of interest can be accumulated in node, embryo sac, flowers and/or fruit. For instance, the promoter of the present invention is particularly useful in driving the node, embryo sac or flower specific transcription of heterologous structural and regulatory genes in plants. In a particularly preferred embodiment, the present invention relates to a DNA construct with a promoter and/or 3′ regulatory element of the present invention operably linked to a coding sequence for a toxic protein specifically inhibiting the formation of a particular flower, embryo sac or node tissue.

In the context of the present invention, a number of terms shall be utilised as follows. The term “promoter” refers to a sequence of DNA, usually upstream (5′) to the coding sequence of a structural gene, which controls the expression of the coding region by providing the recognition for RNA polymerase and/or other factors required for transcription to start at the correct site. Promoter sequences are necessary, but not always sufficient to drive the expression of the gene.

A “3′ regulatory element (or “3′ end”) refers to that portion of a gene comprising a DNA segment, excluding the 5′ sequence which drives the initiation of transcription and the structural portion of the gene, that determines the correct termination site and contains a polyadenylation signal and any other regulatory signals capable of effecting messenger RNA (mRNA) processing or gene expression.

The polyadenylation signal is usually characterised by effecting the addition of polyadenylic acid tracts to the 3′ end of the mRNA precursor. Polyadenylation signals are commonly recognised by the presence of homology to the canonical form 5′-AATAA-3′, although variations are not uncommon.

“Nucleic acid” refers to a large molecule which can be single or double stranded, composed of monomers (nucleotides) containing a sugar, phosphate and either a purine or pyrimidine. The nucleic acid may be cDNA, genomic DNA, or RNA, for instance mRNA.

The term “nucleic acid sequence” refers to a natural or synthetic polymer of DNA or RNA which may be single or double stranded, alternatively containing synthetic, non-natural or altered nucleotide bases capable of incorporation into DNA or RNA polymers.

The term “gene” refers to a DNA sequence that codes for a specific protein and regulatory elements controlling the expression of this DNA sequence.

The term “regulatory element” refers to a sequence located upstream (5′), within and/or downstream (3′) to a coding sequence whose transcription and expression is controlled by the regulatory element, potentially in conjunction with the protein biosynthetic apparatus of the cell. “Regulation” or “regulate” refer to the modulation of the gene expression induced by DNA sequence elements located primarily, but not exclusively upstream (5′) from the transcription start of the gene of interest. Regulation may result in an all or none response to a stimulation, or it may result in variations in the level of gene expression.

The term “coding sequence” refers to that portion of a gene encoding a protein, polypeptide, or a portion thereof, and excluding the regulatory sequences which drive the initiation or termination of transcription.

The coding sequence or the regulatory element may be one normally found in the cell, in which case it is called “autologous”, or it may be one not normally found in a cellular location, in which case it is termed “heterologous”.

A heterologous gene may also be composed of autologous elements arranged in an order and/or orientation not normally found in the cell in which it is transferred. A heterologous gene may be derived in whole or in part from any source known to the art, including a bacterial or viral genome or episome, eukaryotic nuclear or plasmid DNA, cDNA or chemically synthesised DNA. The structural gene may constitute an uninterrupted coding region or it may include one or more introns bounded by appropriate splice junctions. The structural gene may be a composite of segments derived from different sources, naturally occurring or synthetic.

The term “vector” refers to a recombinant DNA construct which may be a plasmid, virus, or autonomously replicating sequence, phage or nucleotide sequence, linear or circular, of a single or double stranded DNA or RNA, derived from any source, in which a number of nucleotide sequences have been joined or recombined into a unique construction which is capable of introducing a promoter fragment and a DNA sequence for a selected gene product in sense or antisense orientation along with appropriate 3′ untranslated sequence into a cell, in particular a plant cell.

As used herein, “plant” refers to photosynthetic organisms, such as whole plants including algae, mosses, ferns and plant-derived tissues. “Plant derived tissues” refers to differentiated and undifferentiated tissues of a plant, including nodes, male and female flowers, fruits, pollen, pollen tubes, pollen grains, roots, shoots, shoot meristems, coleoptilar nodes, tassels, leaves, cotyledondous petals, ovules, tubers, seeds, kernels and various forms of cells in culture such as intact cells, protoplasts, embryos and callus tissue. Plant-derived tissues may be in planta, or in organ, tissue or cell culture. A “monocotyledonous plant” refers to a plant whose seeds have only one cotyledon, or organ of the embryo that stores and absorbs food. A “dicotyledonous plant” refers to a plant whose seeds have two cotyledons.

“Transformation” and “transferring” refers to methods to transfer DNA into cells including, but not limited to, biolistic approaches such as particle bombardment, microinjection, permeabilising the cell membrane with various physical (e.g., electroporation) or chemical (e.g., polyethylene glycol, PEG) treatments; the fusion of protoplasts or Agrobacterium tumefaciens or rhizogenes mediated transformation. For the injection and electroporation of DNA in plant cells there are no specific requirements for the plasmids used. Plasmids such as pUC derivatives can be used. If whole plants are to be regenerated from such transformed cells, there should be a selectable marker. Depending upon the method for the introduction of desired genes into the plant cell, further DNA sequences may be necessary; if, for example, the Ti or Ri plasmid is used for the transformation of the plant cell, at least the right border, often, however, the right and left border of the Ti and Ri plasmid T-DNA have to be linked as flanking region to the genes to be introduced.

If Agrobacteria are used for the transformation, the DNA to be introduced has to be cloned into specific plasmids, either into an intermediary vector or into a binary vector. The intermediary vectors can be integrated into the Ti or Ri plasmid of the Agrobacteria due to sequences that are homologous to sequences in the T-DNA by homologous recombination. The Ti or Ri plasmid furthermore contains the vir region necessary for the transfer of the T-DNA into the plant cell. Intermediary vectors cannot replicate in Agrobacteria. By means of a helper plasmid the intermediary vector can be transferred by means of a conjugation to Agrobacterium tumefaciens. Binary vectors can replicate both in E. coli and in Agrobacteria and they contain a selection marker gene and a linker or polylinker framed by the right and left T-DNA border region. They can be transformed directly into the Agrobacteria (Holsters et al., 1978). The Agrobacterium serving as a host cell should contain a plasmid carrying a vir region. The Agrobacterium transformed is used for the transformation of plant cells. The use of T-DNA for the transformation of plant cells has been extensively examined and described in EP-A 120 516; Hoekema, (1985); An et al., (1985).

For the transfer of the DNA into the plant cell plant explants can be co-cultivated with Agrobacterium tumefaciens or Agrobacterium rhizogenes. From the infected plant material (e.g., pieces of leaf, stem segments, roots, but also protoplasts or plant cells cultivated by suspension) whole plants can be regenerated in a suitable medium, which may contain antibiotics or biozides for the selection of transformed cells.

Alternative systems for the transformation of monocotyledonous plants are the transformation by means of electrically or chemically induced introduction of DNA into protoplasts, the electroporation of partially permeabilised cells, the macroinjection of DNA into flowers, the microinjection of DNA into micro-spores and pro-embryos, the introduction of DNA into germinating pollen and the introduction of DNA into embryos by swelling (Potrykus, (1990)).

While the transformation of dicotyledonous plants via Ti plasmid vector systems with the help of Agrobacterium tumefaciens is well-established, more recent research work indicates that also monocotyledonous plants are accessible for transformation by means of vectors based on Agrobacterium (Chan et al., (1993); Hiei et al., (1994); Bytebier et al., (1987); Raineri et al., (1990), Gould et al., (1991); Mooney et al., (1991); Lit et al., (1992)).

In fact, several of the above-mentioned transformation systems could be established for various cereals: the electroporation of tissues, the transformation of protoplasts and the DNA transfer by particle bombardment in regenerative tissue and cells (Jähne et al., (1995)). The transformation of wheat has been frequently described in the literature (Maheshwari et al., (1995)) and of maize in Brettschneider et al. (1997) and Ishida et al. (1996).

The term “host cell” refers to a cell which has been genetically modified by transfer of a heterologous or autologous nucleic acid sequence or its descendants still containing this sequence. The host cell may be transiently or stably transformed and is preferably able to express the transformed nucleic acid molecule. These cells are also termed “transgenic cells”. In the case of an autologous nucleic acid sequence being transferred, the sequence will be present in the host cell in a higher copy number than naturally occurring.

The term “operably linked” refers to the chemical fusion of two of more fragments of DNA in a proper orientation such that the fusion preserves or creates a proper reading frame, or makes possible the proper regulation of expression of the DNA sequences when transformed into plant tissue.

The term “expression” as used herein is intended to describe the transcription and/or coding of the sequence for the gene product. In the expression, a DNA chain coding for the sequence of gene product is first transcribed to a complementary RNA, which is often an mRNA, and then the thus transcribed mRNA is translated into the above mentioned gene product if the gene product is a protein. However, expression also includes the transcription of DNA inserted in antisense orientation to its regulatory elements. Expression, which is constitutive and possibly further enhanced by an externally controlled promoter fragment thereby producing multiple copies of mRNA and large quantities of the selected gene product, may also include overproduction of a gene product.

A “tissue specific promoter” refers to a sequence of DNA that provides recognition signals for RNA polymerase and/or other factors required for transcription to begin, and/or for controlling expression of the coding sequence precisely within certain tissues or within certain cells of that tissue. Expression in a tissue specific manner may be only in individual tissues, or cells within tissues, or in combinations of tissues. The present invention relates in particular to flower, embryo sac and/or node specific expression, i.e. examples may include tissue specific expression in nodes only and no other tissues within the plant, or may be in nodes and flowers, and no other tissues of the plant. An expression in nodes, embryo sac or flowers according to which the expression takes place mainly, but not exclusively, in the nodes, embryo sac or flowers is also termed “node, embryo sac or flower abundant”.

The term “node, embryo sac or flower specific nucleic acid sequence” refers to nucleic acid sequences, i.e. genes, coding sequences and/or regulatory elements which are exclusively or mainly active in nodes, embryo sac or flowers of plants, in particular those which direct or contribute to a node, embryo sac or flower abundant or selective expression of a protein. The term “node, embryo sac or flower abundant nucleic acid sequence” refers to nucleic acid sequences, i.e. genes, coding sequences and/or regulatory elements which are mainly active in nodes, embryo sacs or flowers of plants, in particular those which direct or contribute to a node, embryo sac or flower abundant expression of a protein.

In a further preferred embodiment the invention relates to nucleic acid molecules specifically hybridising to transcripts of the nucleic acid molecules. These nucleic acid molecules are preferably oligonucleotides having a length of at least 10, in particular of at least 15 and particularly preferred of at least 50 nucleotides. The nucleic acid molecules and oligonucleotides of the present invention may be used for instance as primers for a PCR reaction or be used as components of antisense constructs or of DNA molecules encoding suitable ribozymes.

The present invention also relates to vectors comprising the above-identified nucleic acid molecules in particular comprising chimeric DNA constructs or non-chimeric DNA constructs such as the wild-type ZmMADS3 gene, or derivatives thereof or parts thereof. The term DNA construct refers to a combination of at least one regulatory element and a coding sequence.

Thus, the present invention relates to recombinant nucleic acid molecules useful in the preparation of plant cells and plants as defined above by genetic engineering. In particular, the invention concerns chimeric DNA constructs comprising a coding DNA sequence coding for a wild-type ZmMADS3 protein operably linked to a promoter wherein said promoter is different to the promoter linked to said ZmMADS3 coding sequence in the wild-type gene i.e. either is a mutated wild-type promoter or a promoter form another gene and/or species. In a further preferred embodiment, the invention concerns chimeric DNA constructs comprising a modified coding DNA sequence coding for a mutated ZmMADS3 protein, wherein the DNA-sequence is operably linked to a promoter which may be different from the promoter linked to said ZmMADS3 coding sequence in the wild-type gene or the promoter is the wild-type ZmMADS3 promoter.

Of course, the present invention also relates to chimeric antisense constructs comprising a DNA sequence encoding, at least partially, the natural, that is wild-type, or modified ZmMADS3 protein, or a part thereof, which is linked to a promoter wherein said promoter is different to the promoter linked to said ZmMADS3 coding sequences in the wild-type gene or is the wild-type promoter and wherein the orientation of the coding sequence to the promoter is vice versa to the wild-type orientation. In one embodiment of the present invention the DNA sequence of the present invention used specifically to inhibit via antisense constructs the translation of ZmMADS3 expression from the wild-type gene is at least partially not derived from the ZmMADS3 coding sequence but rather contains sequences from untranslated regions of the ZmMADS3 transcribed region. Both the ZmMADS3 coding sequence and the untranslated region of the ZmMADS3 gene are also termed ZmMADS3 derived sequence. of course the invention also relates to DNA constructs comprising a DNA sequence coding for the non-chimeric wild-type ZmMADS3 protein operably linked to the wild-type promoter. These constructs may be used to transform plant cells and plants for which the DNA construct is autologous, i.e. is the source or natural environment for the DNA construct or for which the DNA construct is heterologous, i.e., is from another species. Plant cells and plants obtained by using the above listed DNA constructs may be characterised by ZmMADS3 antisense expression, multiple copies of the above DNA constructs in their genome, that means are characterised by an increased copy number of the ZmMADS3 gene in the genome and/or a different location in the genome with respect to the wild-type gene and/or the presence of a foreign gene in their genome.

In the context of the present invention a chimeric DNA construct is thus a DNA sequence composed of different DNA fragments not naturally occurring in this combination. The DNA fragments combined in the chimeric DNA construct may originate from the same species or from different species. For example a DNA fragment coding for an ZmMADS3 protein may be operably linked to a DNA fragment representing a promoter from another gene of the same species that provides for an increased expression of the ZmMADS3 coding sequence. Preferably however, a DNA fragment coding for an ZmMADS3 protein is operably linked to a DNA fragment containing a promoter from another species for instance from another plant species, from a fungus, yeast or from a plant virus or a synthetically produced promoter. A synthetically produced promoter is either a promoter synthesised chemically from nucleotides de novo or a hybrid-promoter spliced together by combining two or more nucleotide sequences from synthetic or natural promoters which are not present in the combined form in any organism. The promoter has to be functional in the plant cell to be transformed with the chimeric DNA construct.

The promoter used in the present invention may be derived from the same or from a different species and may provide for constitutive or regulated expression, in particular positively regulated by internal or external factors. External factors for the regulation of promoters are for example light, heat, chemicals such as inorganic salts, heavy metals or organic compounds such as organic acids, derivatives of these acids, in particular its salts.

Examples of promoters to be used in the context of the present invention are the actin promoter from rice, the cauliflower mosaic virus (CaMV) 19S or 35S promoters, nopaline synthase promoters, pathogenesis-related (PR) protein promoters, the ubiquitin promoter from maize for a constitutive expression, the HMG promoters from wheat, promoters from Zein genes from maize, small subunit of ribulose bisphosphonate carboxylase (ssuRUBISCO) promoters, the 35S transcript promoter from the fig-worm mosaic virus (FMV 35S), the octopine synthase promoter etc. It is preferred that the particular promoter selected should be capable of causing sufficient expression to result in the production of an effective amount of antisense mRNA or modified or wild-type ZmMADS3 protein to produce flower and/or fruit modified plants. Of course for selective expression of the ZmMADS3 protein tissue specific promoters may be used. However, in the most preferred embodiment of the present invention, i.e. the ZmMADS3 antisense constructs, the promoter may be a constitutive strong promoter, since the node or flower specificity of the antisense action is confined to the nodes or flowers due to node or flower specific expression of the target, i.e. the wild-type ZmMADS3 expression.

The DNA construct of the invention may contain multiple copies of a promoter and/or multiple copies of the DNA coding sequences. In addition the construct may include coding sequences for markers and coding sequences for other peptides such as signal or transit peptides or resistance genes for instance against virus infections or antibiotics.

Useful markers are peptides providing antibiotic or drug resistance for example resistance to phosphin-strycine, hygromycin, kanamycin, G418, gentamycin, lincomycin, methotrexate or glyphosate. These markers can be used to select cells transformed with the chimeric DNA constructs of the invention from untransformed cells. Thus, a useful master gene is the herbicide resistance gene Pat (phosphinotrycine acetyl transferase). Of course other markers are markers coding peptidic enzymes which can be easily detected by a visible reaction for example a colour reaction for example luciferase, β-1,3-glucuronidase or β-galactosidase.

Signal or transit peptides provide the ZmMADS3 protein formed on expression of the DNA constructs of the present invention with the ability to be transported to the desired site of action. Examples for transit peptides of the present invention are chloroplast transit peptides or mitochondria transit peptides, especially nuclear recognition/localisation signal peptides.

In chimeric DNA constructs containing coding sequences for transit peptides these sequences are usually derived from a plant, for instance from corn, potato, Arabidopsis or tobacco. Preferably, transit peptides and ZmMADS3 coding sequences are derived from the same plant, for instance corn. In particular such a chimeric DNA construct comprises a DNA sequence coding for a wild-type ZmMADS3 protein and a DNA sequence coding for a transit peptide operably linked to a promoter wherein said promoter is different to the promoter linked to said coding sequences in wild-type gene, but functional in plant cells. In particular, said promoter provides for higher transcription efficiency than the wild-type promoter.

The mRNA produced by a DNA construct of the present invention may advantageously also contain a 5′ non-translated leader sequence. This sequence may be derived from the promoter selected to express the gene and can be specifically modified so as to increase translation of the mRNA. The 5′ non-translated regions can also be obtained from viral RNAs from suitable eucaryotic genes or a synthetic gene sequence.

Preferably, the coding sequence of the present invention is not only operably linked to 5′ regulatory elements, such as promoters, but is additionally linked to other regulatory elements such as enhancers and/or 3′ regulatory elements. For instance, the vectors of the present invention may contain functional terminator sequences such as the terminator of the octopine synthase gene from Agrobacterium tumefaciens. Further 3′ non-translated regions to be used in a chimeric construct of the present invention to cause the addition of polyadenylate nucleotides to the 3′ end of the transcribed RNA are the polyadenylation signals of the Agrobacterium tumefaciens nopaline synthase gene (NOS) or from plant genes like the soy bean storage protein gene and the small subunit of the ribulose-1,5-bisphosphonate carboxylase (ssuRUBISCO) gene. of course, also the regulating elements of the present invention deriving from the wild-type ZmMADS3 gene may be used.

The vectors of the present invention may also possess functional units effecting the stabilisation of the vector in the host organism, such as bacterial replication origins. Furthermore, the chimeric DNA constructs of the present invention may also encompass introns or part of introns inserted within or outside the coding sequence for the ZmMADS3 protein.

In a particularly preferred embodiment of the present invention the vector furthermore contains T-DNA, in particular the left, the right or both T-DNA borders derived from Agrobacterium tumefaciens. Of course, sequences derived from Agrobacterium rhizogenes may also be used. The use of T-DNA sequences in the vector of the present invention enables the Agrobacterium mediated transformation of cells. In a preferred embodiment of the present invention the nucleic acid sequence of the present invention, optionally operably linked to regulatory elements, is inserted within the T-DNA or adjacent to it.

Furthermore, the present -invention relates to a wild-type or modified ZmMADS3 protein coded by a nucleic acid sequence of the present invention. The ZmMADS3 protein exhibits in a particularly preferred embodiment features of a MADS-box protein and in particular transcriptional regulative activity during flower and/or fruit development, in particular flower and/or fruit growth, and function. In particular, the present invention relates to a ZmMADS3 protein produced from a plant cell or plant of the present invention or from the propagation material or harvest products of plants or plant cells of the present invention. The invention also relates to antibodies, in particular mono- or polyclonal antibodies raised against the protein with the activity of an ZmMADS3 protein which may be useful for cloning and detection assays. In the context of the present invention, the activity of a ZmMADS3 protein is defined as the activity of an transcriptional activator or repressor of genes needed for flower organ development, embryo, endosperm and seed development.

Thus, the present invention also relates to a method for the production of a protein with the activity of an ZmMADS3 protein, wherein a cell of the present invention, in particular a plant cell or plant callus is cultivated under conditions allowing the synthesis of the protein and the protein is isolated from cultivated cells and/or the culture medium.

In a particularly preferred embodiment of the present invention the 5′ and/or 3′ regulatory elements of the present invention contained in the vector are operably linked to a gene of interest which in this context may also be only its coding sequence, which may be a heterologous or autologous gene or coding sequence. Such a gene of interest may be a gene, in particular its coding sequence, conferring, for instance, apomixis; disease resistance; drought resistance; insect resistance; herbicide resistance; immunity; an improved intake of nutrients, minerals or water from the soil; or a modified metabolism in the plant, particularly in its flower and/or fruits. In the context of the present invention the term apomixis refers to asexual reproduction through seeds. Such a modified metabolism may relate to a preferred accumulation of useful or toxic substances in flowers and/or fruits, for instance sugars, proteins, fats or pigments or, vice versa, in the depletion of substances undesirable in flowers and/or fruits, for instance certain amino acids. Thus, in the context of the present invention, a gene of interest may confer resistance to infection by a virus, such as a gene encoding the capsid protein of the BWYV or the BNYVV virus, a gene conferring resistance to herbicides such as Basta®, or to an insecticide, a gene conferring resistance to the corn rootworm, a gene encoding the toxic crystal protein of Bacillus thuringiensis or a gene whose expression confers male and/or female sterility. A gene of interest includes also a coding sequence cloned in antisense orientation to the regulatory sequences directing its expression. Such an antisense-construct may be used specifically to repress the activity of undesirable genes in plant cells, in particular in flower and/or nodes, for instance to produce plants exhibiting a modified fruit or flower development, metabolism and/or modified function. The gene of interest may also comprise signal sequences, in particular ER targeting sequences, directing the encoded protein in the ER and eventually for instance in the cell wall, vascular tissue and /or the vacuole.

Thus, the nucleic acid sequences of the present invention are also useful since they enable the node, flower and/or fruit specific expression of a ZmMADS3 derived sequence in antisense orientation so as to hybridise to naturally expressed ZmMADS3 transcripts and of further genes of interest in plants, in particular monocotyledonous plants. Thus, also ZmMADS3 target genes are switched on or off. Accordingly, plants are enabled to produce useful products in their flowers and/or fruits or the plants may be engineered by modifying their structure, function and/or development. Plants may be obtained having an increased number of fruits/seeds, for instance corn with two cobs and/or increased ovary numbers.

The present invention also relates to a method of genetically modifying a cell by transforming it with a nucleic acid molecule of the present invention or vector according to the above, whereby the ZmMADS3 coding sequence or a further gene of interest operably linked to at least one regulatory element either according to the present invention or as conventionally used is expressible in the cell. In particular, the cell being transformed by the method of the present invention is a plant, bacterial or yeast cell. In a particularly preferred embodiment of the present invention, the above method further comprises the regeneration of the transformed cell to a differentiated and, in a preferred embodiment, fertile or non-fertile plant.

The present invention also relates to host cells transformed with the nucleic acid molecule or the vector of the present invention, in particular plant, yeast or bacterial cells, in particular monocotyledonous or dicotyledonous plant cells. The present invention also relates to cell cultures, tissue, calluses, etc. comprising a cell according to the above, i.e. a transgenic cell and its descendants harbouring and preferably experiencing the nucleic acid molecule or vector of the present invention.

Thus, the present invention relates to transgenic plant cells which were transformed with one or several nucleic acid molecules of the present invention as well as to transgenic plants cells originating from such transformed cells. Such plant cells can be distinguished from naturally occurring plant cells by the observation that they contain at least one nucleic acid molecule according to the present invention which does not naturally occur in these cells, or by the fact that such a molecule is integrated into the genome of the cell at a location where it does not naturally occur, that is, in another genomic region, or by the observation that the copy number of the nucleic acid molecules is different from the copy number in naturally occurring plants, in particular a higher copy number.

Thus, the present invention also relates to transgenic cells, also called host cells, transformed with the nucleic acid molecule or vector of the present invention, in particular plant, yeast or bacterial cells, in particular monocotyledonous or dicotyledonous plant cells. The present invention also relates to cell cultures, tissue, fruits, flowers, calluses, propagation and harvest material, pollen, seeds, stamen, cobs, nodes, seedlings, somatic and zygotic embryos, etc. comprising a cell according to the above, that is, a transgenic cell being stably or transiently transformed and being capable of expressing a nucleic acid sequence for encoding a protein modifying the flower, seed and/or fruit development of the transformed plant. The transgenic plants of the present invention can be regenerated to whole plants according to methods known to the person skilled in the art. The regenerated plant may be chimeric with respect to the incorporated foreign DNA. If the cells containing the foreign DNA develop into either micro- or macro-spores the integrated foreign DNA will be transmitted to a sexual progeny. If the cells containing the foreign DNA are somatic cells of the plant, non-chimeric transgenic plants are produced by conventional methods of vegetative propagation either in vivo, i.e. from buds or stem cuttings or in vitro following established procedures known in the art.

Thus, the present invention also relates to transgenic plants, parts of plants, plant tissue, reproductive and vegetative tissue, plant seeds, plant embryos, plant seedlings, plant propagation material, plant harvest material, plant leaves and plant pollen, stamen, cobs, nodes, fruits, flowers, plant roots containing the above identified plants cell of the present invention. These plants or plant parts are characterised by, as a minimum, the presence of the heterologous transferred DNA construct of the present invention in the genome or, in cases where the transferred nucleic acid molecule is autologous to the transferred host cell are characterised by additional copies of the nucleic acid molecule of the present invention and/or a different location within the genome. Thus, the present invention also relates to plants, plant tissues, plant reproductive and vegetative tissue, plant seeds, plant seedlings, plant embryos, propagation material, harvest material, leaves, nodes, cobs, stamen, fruits, flowers, pollen, roots, calluses, tassels etc. non-biologically transformed which possess stably or transiently integrated in the genome of the cells, for instance in the cell nucleus, plastids or mitochondria a heterologous and/or autologous nucleic acid sequence containing (a) a coding sequence of the present invention or (b) a regulatory element of the present invention recognised by the polymerases of the cells of the said plant and, in a preferred embodiment, being operably linked in sense or antisense orientation to in case of (a) at least one regulatory element or in case of (b) a coding sequence of a gene of interest. The teaching of the present invention is therefore applicable to any plant, plant genus or plant species wherein the regulatory elements mentioned above are recognised by the polymerases of the cell. Thus, the present invention provides plants of many species, genuses, families, orders and classes that are ably to recognise these regulatory elements of the present invention or derivatives or parts thereof.

Any plant is considered, in particular plants of economic interest for example plants grown for human or animal nutrition, plants grown for the content of useful secondary metabolites, plants grown for their content of fibres, trees and plants of ornamental interest. Examples which do not imply any limitation as to the scope of the present invention are corn, wheat, barley, rice, sorghum, sugarcane, sugarbeet, soybean, Brassica, sunflower, carrot, tobacco, lettuce, cucumber, tomato, potato, cotton, Arabidopsis, Lolium, Festuca, Dactylis, or poplar.

The present invention also relates to a process, in particular a microbiological process and/or technical process, for producing a plant or reproduction material of said plant, including an heterologous or autologous DNA construct of the present invention stably or transiently integrated therein, and capable of being expressed in said plants or reproduction material, which process comprises transforming cells or tissue of said plants with a DNA construct containing a nucleic acid molecule of the present invention, i.e. a regulatory element which is capable of causing the stable integration of the ZmMADS3 derived sequences in particular a coding sequence in said cell or tissue and enabling the sense or antisense expression of a ZmMADS3 derived sequence, in particular coding sequence or part thereof in said plant cell or tissue, regenerating plants or reproduction material of said plant or both from the plant cell or tissue transformed with said DNA construct and, optionally, biologically replicating said last mentioned plants or reproduction material or both. The present invention also relates to the above process, wherein instead or in addition to the ZmMADS3 derived, in particular coding sequence, a regulatory element of the ZmMADS3 gene of the present invention is transformed into a plant, preferably operably linked to a coding sequence of interest.

Finally, the present invention relates to a method for isolating or cloning flower, embryo sac and/or fruit specific genes and/or the corresponding specific regulatory elements, such as promoters, or MADS box genes whereby a nucleic acid sequence of the present invention is used to screen nucleic acid sequences derived from any source, such as genomic or cDNA libraries derived from plants, in particular monocotyledonous plants. The nucleic acid sequences of the present invention thereby provide a means of isolating related regulatory sequences of other plant species which confer flower or fruit specificity to genes of interest operably linked to them.

Further preferred embodiments of the present invention are mentioned in the subclaims.

The invention may be more fully understood from the following detailed sequence descriptions which are part of the present teaching. The SEQ ID Nos. 1 to 18 are incorporated in the present invention.

SEQ ID No. 1 represents the complete cDNA-sequence of the ZmMADS3 (Zea mays MADS-box) gene.

SEQ ID No. 2 represents the amino acid sequence of the ZmMADS3 protein.

SEQ ID Nos. 3 to 21 represent primers used for cloning and detecting nucleic acid sequences of the present invention and/or transcripts expressed thereby.

The invention is further illustrated by way of example and the following drawings.

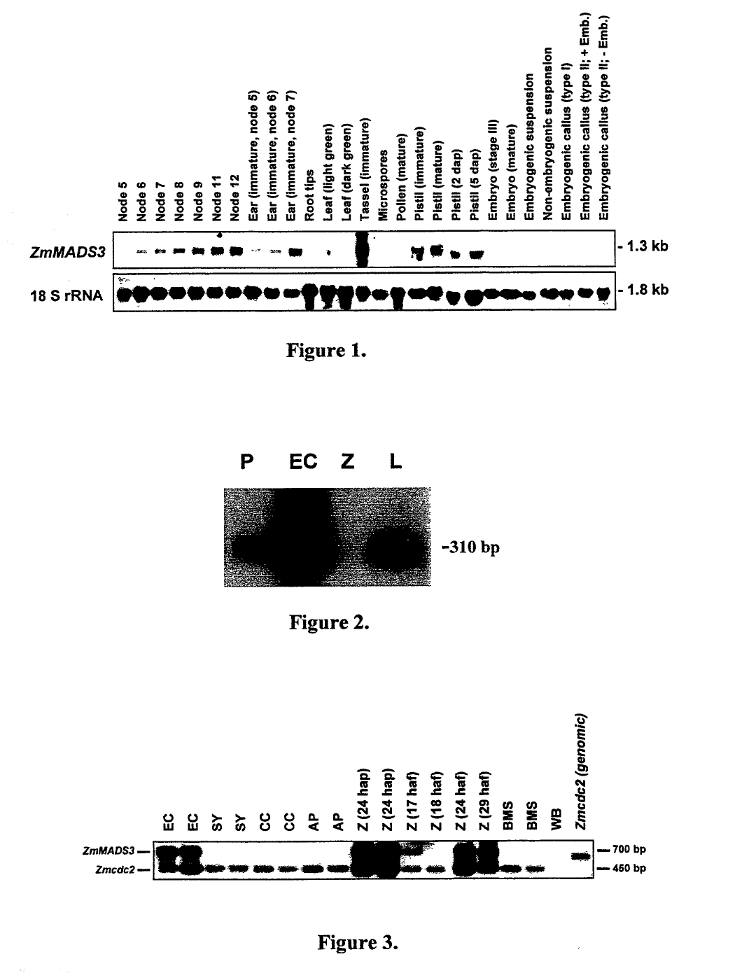

FIGS. 1 to 4 show expression analyses of ZmMADS 3 in various tissues of Zea mays with gene specific hybridisation conditions.

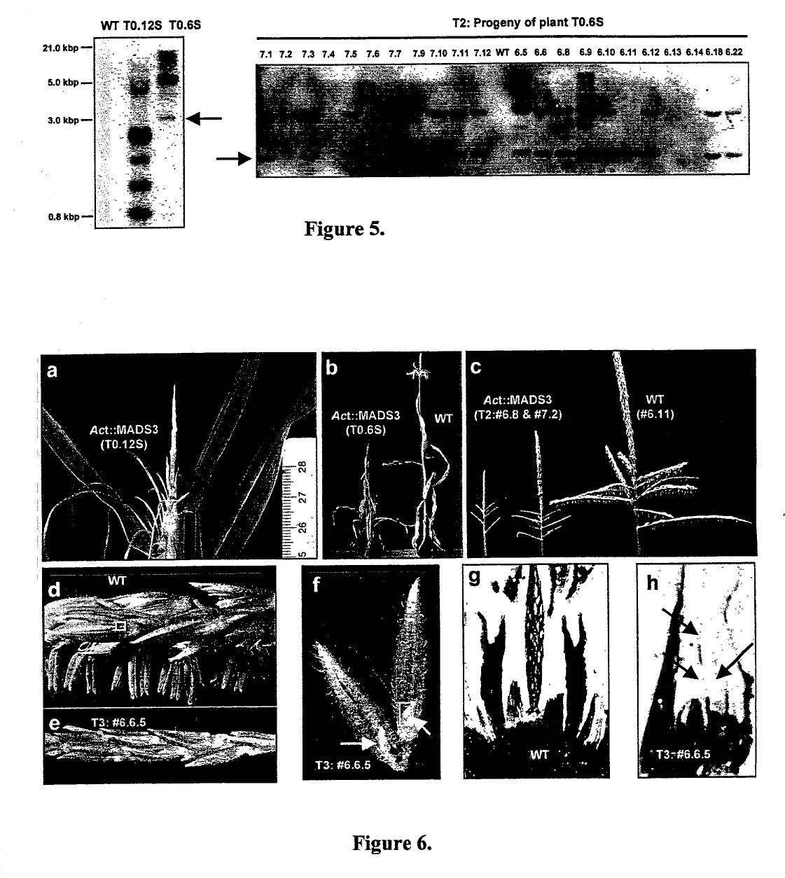

FIG. 5 shows the integration of the full length construct in Sense-plants.

FIG. 6 shows the phenotype of Sense-plants.

### EXAMPLE 1

### Cloning of the ZmMADS3 cDNA Sequence

Plant Material and Pollen Isolation

Tissues were isolated from Zea mays L. inbred line A188 (Green and Philips, 1975) cultivated in a greenhouse. Embryos from kernels (12 dap and mature) were isolated under sterile conditions. Seedlings were germinated under sterile conditions in the dark and were dissected into cotyledons, roots tips and scutella. For isolation of pollen before anthesis, tassels were divided into upper (mature stage) and lower parts (immature stage). Pollen was isolated as described by Mordhorst and Lörz (1993) and different developmental stages were separated via a discontinuous Percoll gradient (20%, 30% and 40% Percoll in 0.4 M mannitol). Centrifugation was performed for 10 min at 20° C. and 226×g in a swing out rotor with slow acceleration and deceleration. Developmental stages of pollen were monitored microscopically and by DAPI staining.

RNA Isolation and Construction of cDNA Libraries

Total RNA was isolated from various tissues with TRIzol (GibcoBRL). Seasand was added for the maceration of pollen. Total RNA was isolated from mature pollen for the construction of a cDNA library using the protocol described by Stirn et al. (1995). The library was constructed from 5 μg poly(A)⁺ RNA as outlined by Dresselhaus et al. (1996b) using the Uni-ZAP XR lambda vector (Stratagene). Total RNA from leaf material of 10 day old seedlings was isolated as described by Logemann et al. (1987) and a cDNA library was generated from 2 μg poly(A)⁺ RNA (seedlings library).

cDNA libraries from egg cells and zygotes of maize were generated as described by Dresselhaus et al. 1994, 1996a.

Screening of cDNA Library with Maize MADS Box probes

The highly conserved MADS box of different maize MADS box genes was amplified from the maize genome by PCR and served as probes for the plaque screening of a cDNA library of mature maize pollen, egg cells and zygotes.

Genomic DNA from leaf material was isolated as outlined by Dellaporta et al. (1983) and served as template for the synthesis of different MADS box probes. MADS box gene specific primers with the nucleotide sequence specified in SEQ ID No. 3 to 14 were used to specifically amplify the MADS box region of maize MADS box genes:

ZMM1 (5′-ATGGGGAGGGGAAGGATTGA-3′, SEQ ID No. 3; 5′-CTGTTGTTGGCGTACTCGTAG-3′, SEQ ID No. 4),

ZEM2/3/ZAG 4 (5′-AGGGGCAAGATCGACATCAAG-3′, SEQ ID No. 5;

5′-GG/TCGT/AACTCGTAGAGGCGG-3′, SEQ ID No. 6), ZAG3/5 (5′-ATGGGGAGGGGACGA/CGTTGA-3′, SEQ ID No. 7;

5′-GCTGCCGAACTCGTAGAGCT-3′; SEQ ID No. 8), ZAP1 (5′-GTTGTTGGCGTACTCGTAGAG-3′, SEQ ID No. 9;

5′-GGGCGCAAGGTACAGCTGAA-3′, SEQ ID No. 10), ZAG1 (5′-GTTGTTGGCGTACTCGTAGAG-3′, SEQ ID No. 11;

5′-AAGGGCAAGACTGAGATCAAG-3, SEQ ID No. 12) and ZAG2 (5′-CACTTGAACTCTTTTACGCTTAT-3′, SEQ ID No. 13;

5′-GACAATCTTGACACATGTATGAA-3′, SEQ ID No. 14);

Amplification of the MADS box and flanking genomic regions: PCR amplification was performed with 200 ng genomic DNA in a standard reaction mixture: 250 nM primer, 2 mM MgCl₂, 400 μM dNTPs and 1.25 U Taq DNA polymerase (GibcoBRL) in PCR buffer (50 mM KCl, 20 mM Tris-HCl, pH 8.4). Hot start PCRs were performed with the following profile: 5 min 95° C., 3 min 75° C. (addition of Taq-DNA polymerase) followed by 30 cycles with 1 min 96° C., 1 min 62° C. (ZEM, ZMM, ZAG3) or 60° C. (ZAG1, ZAP1) and 3 min 72° C. A final extension was performed for 5 min at 72° C. PCR products were separated on low melting agarose gels (NuSieve GTG, BIOzym) and isolated gel fragments containing the DNA's were digested with Gelase (BIOzym). Probes were labelled with [³²P]-dCTP (6000 Ci/mmol), Amersham) using the Prime-it II random primer labelling Kit (Stratagene) and purified with NucTrap columns (Stratagene). Approximately 22.000 phages from each of the pollen, egg cell and zygote libraries were plated per 15 cm plate and transferred by Hybond-N membranes (Amersham) as double plague lifts according to Sambrook et al. (1989). Prehybridisation was performed with 50 μg/ml salmon sperm DNA in hybridisation buffer (5×SSPE, 5× Denharts, 0.5% SDS) for 5 h at 55° C. Filters were hybridised with a cocktail of the different MADS box probes in a final concentration of 650.000 cpm for each probe/ml hybridisation buffer. After hybridisation overnight at 55° C., filters were washed three times for 15 min with 5×SSPE/0.1% SDS and exposed to X-Omat AR films (Amersham) using intensifier screens at −70° C. Putative positive lambda phages were isolated and cDNAs excised according to the manufacturer (ZAP-cDNA Synthesis Kit, Stratagene).

Thus, approximately 250.000 phages were screened with the MADS box probes at medium stringent conditions to permit hybridisation to less homologous sequences. Thirteen putative positive signals were analysed further and one cDNA coding a protein with high homology to MADS box proteins was isolated, designated ZmMADS3.

DNA Sequencing and Sequence Analysis

Sequencing of the cDNA was performed with the ABI PRISM Dye Terminator Kit with TaqFS DNA polymerase (PE Applied Biosystems) according to the manufacturers protocol, except that 800 ng of template DNA and 5 pmole vector primers were used. Sequence analyses were performed with DNASIS 1.1 software program package (HITACHI).

The cDNA of ZmMADS3 is 1250 bp in length with an open reading frame of 270 amino acids. The 3′-UTR is 368 bp in length. Calculated from the 5′ cDNA end, the 5′-UTR spans from position 1 to 69.

The sequence of the full-length cDNA is given in SEQ ID No. 1. The amino acid sequence of ZmMADS3 is given in SEQ ID No. 2. ZmMADS3 contains a MADS-box at the N-terminal end consisting of 57 amino acids. The MADS-box is followed by a linker region of 35 amino acids and a K-box comprising 66 amino acids. A putative bipartite nuclear localisation signal (KR-(X)₁₂-KRR) is located in the MADS box of ZmMADS3. The bipartite signal motive is comprised of two basic amino acids and a spacer of 12 variable amino acids.

### EXAMPLE 3

### Northern Blot and PCR Analyses

Ten μg of total RNA extracted from various tissues were separated on denaturating agarose gels and transferred to Hybond N⁺ membranes (Amersham) by capillary blotting with 10× SSC overnight. The RNA was fixed to the membrane by UV crosslinking with 300 mJoule in a Stratalinker 1800 (Stratagene). The filters were pre-hybridised for 5 hours at 65° C. with 100 μg/ml HS-DNA in CHURCH-Puffer (0.5 M NaH₂PO₄ (pH 7.2), 7% SDS, 1 mM EDTA). After overnight hybridisation with a probe concentration of 10⁶cpm/ml the filters were washed a total of 6 times for 15 minutes at 65° C. with decreasing SSC concentration (2×, 1×, 0.5× and 0.2×SSC, 0.1% SDS; 2×0.1×SSC, 0.1% SDS; Sambrook et al., 1989). The exposition of the filters on X.Omat AR films (Amersham) took place in cassettes with reinforced film at −70° C. The size of the RNA was determined by use of the RNA GibcoBRL size standard 0.24-9.5 kb.

Gene specific probes were amplified from plasmids containing ZmMADS3 cDNA with primers specific for the 3′-end of ZmMADS3 (SEQ. ID No. 15 and SEQ. ID 16; UTR for and UTR rev). Expression of ZmMADS3 in single egg cells was detected according to Richert et al. (1996) and with cDNA libraries described above using gene specific primers UTR for and UTR rev. (SEQ. ID. Nos. 15 and 16).

FIG. 1 shows Northern Blot analyses of ZmMADS3.

10 μg total RNA of each given tissue was electrophoretically separated on denaturated agarose gel, blotted and hybridised with a ³²p-labelled ZmMADS3-specific probe from the 3′ untranslated region. The exposition after two weeks is shown. The size of the band is indicated (kb, kilo base). The ribosomal RNAs are shown as a control. ZmMADS3 mRNA was detected primarily in nodes, immature flower organs and pistils before and after fertilization.

FIG. 2 indicates the presence of ZmMADS3 in cDNA-libraries of egg cells (EC), zygotes (Z), leaves (L) of seedlings and pollen (P).

The cDNA libraries of pollen, egg cells, in vitro zygotes (18 h after in vitro fertilization) and leaves of seedlings were examined with UTR for and UTR rev gene specific primers (SEQ ID No's 15 and 16) in the presence of ZmMADS3 cDNA. PCR-fragments were gel-electrophoretically separated, blotted and hybridised with ³²P-labelled gene specific probes. The size of the bands is indicated (bp base pairs).

FIG. 3 shows the results of single cell RT-PCR with isolated embryo sac cells and zygotes.

Single, isolated cells of the embryo sac and zygotes at different stages after in vivo and in vitro fertilization were analysed, without prior RNA isolation, for the expression of ZmMADS3 in RT-PCR experiments after Richert et al. (1996). The RNA was transcribed into cDNA with SEQ ID No. 16, amplified by PCR with SEQ ID No's 16 and 19 and gel-electrophoretically separated. A cdc2 gene from maize was reverse transcribed and PCR amplified with the primers 5′-ACTCATGAGGTAGTGACATT-3′ (SEQ ID No. 20) and 5′-CATTTAGCAGGTCACTGTAC-3′ (SEQ ID No. 21) and served as a control for the success of the RT-PCR experiment (multiplex-RT-PCR). The size of the bands is indicated. In the unfertilized embryo sac, ZmMADS3 is exclusively expressed in the egg cell and after fertilization in both, in vivo and in vitro zygotes. (bp: base pairs; EC: egg cell; CC: central cell; SY: synergide; AP: 15 antipodal cells; Z: zygote; BMS: suspension cell; WB: washing buffer; hap: h after in vitro pollination; haf: h after fusion).

### EXAMPLE 4

### In Situ Hybridization Analysis

Digoxigenin-labeled RNA probes were synthesized from ZmMADS3 gene specific 3′-end (see above), which was cloned into pGEM-T-vector (Promega). Probes were synthesized from 1 μg plasmid at 37° C. for 3-4 h in 40 μl assays (40 U T7 or Sp6 RNA polymerase (Boehringer), 4 μl NTP labeling mix (Boehringer), 20 U RNasin (Promega) according to the manufacturer's protocol (Boehringer). Male and female flowers of various developmental stages were collected from Zea mays inbred line A188 and B73. In situ hybridization procedure essentially followed the protocol provided by Canas et al. (1993). Samples were fixed in EAF-Medium (50% ethanol, 5% acetic acid and 4% paraformaldehyd) and embedded in paraffin (Paraplast Plus, Sigma). 8-10 μm sections were digested with 1 μg/ml Proteinase K (Boehringer) for 30 min at 37° C. Further treatment and hybridization to gene specific probes was performed as described by Canas et al. (1993).

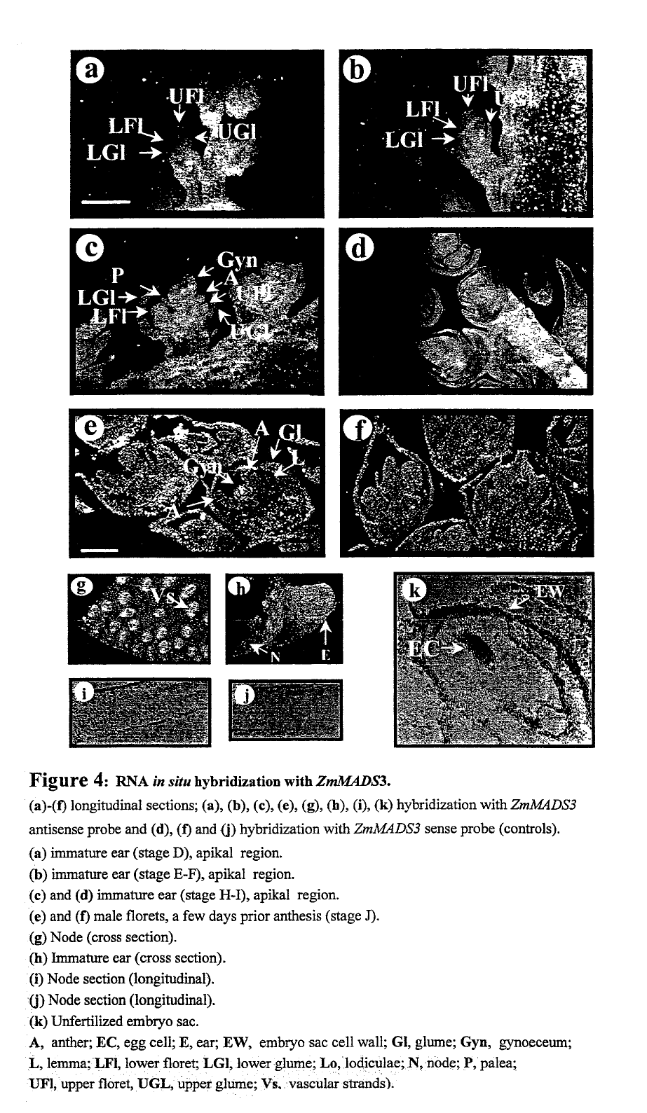

FIG. 4 shows the result of RNA in situ hybridization analysis.

ZmMADS3 is expressed in all immature male and female flower organ meristems. ZmMADS3 is further expressed in the basal meristematic cells in nodes, and in the unfertilized embryo sac, ZmMADS3 expression was detected in the egg cell only. Flower organs are described below FIG. 4.

### EXAMPLE 5

### Transformation and Regeneration of ZmMADS3 Transgenic Plants

Transgenic Plants

The vector pAct1.cas (Biorad, Munich) was used for the cloning of sense- and antisense-constructs of ZmMADS3.

In order to prepare antisense-constructs, cleavage sites for the restriction enzyme KpnI and HindIII were introduced into the gene-specific 3′-end of ZmMADS3 by means of PCR. The amplification took place with ZmMADS3 specific primers AS1/KpnI (SEQ ID No. 17) and AS2.3/HindIII (SEQ. ID No. 18) in a PCR with the following profile: 30 cycles 20 s 96° C., 2 min. 58° C., 2.5 min. 72° C. The DNA fragment and the vector pAct1.cas were digested with HindIII and KpnI according to the manufacturers instructions (Gibco BRL) and purified before cloning on a 1% low-melting agarose gel (LM-agarose, NuSieve GTG, BIOzym). The position of the HindIII and KpnI cleavage sites in the vector cause the DNA-fragment to integrate into the vector in antisense-orientation. In order to prepare the sense-constructs, ZmMADS3 plasmid (complete cDNA cloned in lambda Uni-ZAP XR into the EcoRI/XhoI cleavage sites) and the pAct1.cas vector were digested with the restriction enzymes SmaI and KpnI in accordance with the manufacturers instructions (Gibco BRL) and purified on a LM-gel (see above). As the cleavage sites for SmaI and KpnI are located in the polylinker of the lambda Uni-ZAP XR, this digestion caused the complete ZmMADS3 cDNA with short, flanking vector sequences to be cut out of the polylinker. The ligation was achieved over night with RT (antisense construct) or 6 hours at 26° C. (sense-construct) with T4-DNA ligase according to the manufacturers instructions (Gibco BRL).

Carrying out the Transformation and Tissue Cultures

Immature embryos (12 days after pollination) of the inbred A188 strain and of crosses of the A188×H99 strain were used. The seeds' surface was sterilised for 20 min in 1% sodium hypochloride solution (with 0.1% Mucasol) before isolation and subsequently rinsed three times with sterile H₂O before the embryos were isolated from the seeds under sterile conditions.

Embryos were pre-cultivated for 7-11 days on N6*-medium (callus induction medium, see below) (scutellum facing upwards) and transferred to N6*-osmotic medium 4-6 hours before bombardment; see Brettschneider et al., (1997) below. Plasmids from the sense or antisense constructs of ZmMADS3 in combination with the 35-S-Pat plasmid (Becker et al., (1994)) as selection markers were fixed to gold microcarriers and used for the biolistic transformation of the embryos: 2.5 μg plasmid ZmMADS3 sense construct or antisense construct and 2.5 μg 35-S-Pat plasmid were added to 50 μl 0.4-1.2 μm gold particles (Hereaus [50 mg/ml]). Immediately after the addition of 20 μl spermidin free base [0.1 M] and 50 μl CaCl2 [2.5 M] the probes were vortexed and subsequently centrifuged. The pellet was washed in 250 μl 100% ethanol and, after a further centrifuging, suspended in 240 μl 100% ethanol once again. 3.5 μl was pipetted on macro-carriers and inserted in a PDS 1000/He Gun (BioRad) (pressure: 1350 Psi, Vacuum: 28 Hg/inch, position of the disc: level 4, rupture disk switch: level 2) to bombard the embryos. The embryos were bombarded twice. The transformed embryos were incubated overnight at 26° C. in the dark and transferred to N6*-medium on the following day.

After 7-17 days the calli were transferred to N6*-selection medium (5.0 mg/l PPT) and incubated in the dark at 26° C. for 15-27 days. The calli were transferred to a fresh medium after approximately two weeks (dead areas were removed and large calli were divided).

After transfer of the calli onto MS-medium (2.5 mg/l PPT) the dishes were transferred to light (16 hours light, 8 hours dark, 24° C.) and cultivated on this medium until shoots and roots were formed. Young plants were transferred to magenta trays with ½ MS-medium for further cultivation and finally transferred to a greenhouse. Several weeks after transfer the plants were sprayed a total of three times at several day intervals with a BASTA-solution (250 mg/l PPT, 0.1% Tween 20). Plants that were still green after this spray test were analysed further.

Media used:

| N6 basic medium | | |
|---|---|---|
| N6-macrosalt | 100 | ml/l |
| N6-microsalt | 1 | ml/l |
| N6 vitamin | 1 | ml/l |
| Inositole | 100 | mg/l |
| Fe/Na-EDTA | 2 | ml/l |
| Casamino acids | 100 | mg/l |
| Proline | 0.69 | g/l |
| MgCl₂ × 6 H₂O | 0.75 | g/l |
| MES | 0.5 | g/l |
| Sucrose | 20 | g/l |
| pH 5.8 | | |
| N6* medium | | |
| N6 medium with 1 mg/l 2-4-D | | |
| N6* osmotic medium | | |
| N6* medium with 0.7 M sucrose | | |
| N6* selective medium | | |
| N6* medium without casamino acids with phosphino- | | |
| tricine (PPT) in various concentrations | | |
| N6 macrosalts | | |
| KNO₃ | 28.3 | g/l |
| (NH₄)₂SO₄ | 4.63 | g/l |
| CaCl₂ × 2 H₂O | 1.66 | g/l |
| MgSO₄ × 7 H₂O | 1.85 | g/l |
| KH₂PO₄ | 4.0 | g/l |
| N6 microsalts | | |
| H₃BO₄ | 1.6 | g/l |
| MnSO₄ × H₂O | 3.87 | g/l |
| ZnSO₄ × 7 H₂O | 1.5 | g/l |
| KJ | 0.8 | g/l |
| N6 vitamins | | |
| Glycine | 2.0 | g/l |
| Thiamine-HCl | 1.0 | g/l |
| Pyridoxine-HCl | 0.5 | g/l |
| Nicotinic acid | 0.5 | g/l |
| Na/Fe-EDTA-solution | | |
| Na₂EDTA | 3.73 | g/200 ml |
| FeSO₄ × 7H₂O | 2.78 | g/200 ml |
| MS basic medium | | |
| (Murashige & Skoog, | | |
| 1962) | | |
| MS-macrosalts | 100 | ml/l |
| MS-microsalts | 1 | ml/l |
| MS-vitamins | 1 | ml/l |
| Inositole | 100 | mg/l |
| Fe/Na-EDTA | 2 | ml/l |
| Sucrose | 30 | g/l |
| 2-4-D | 1 | mg/l |
| pH 6.0 | | |
| MS-Medium | | |
| MS-medium without 2-4-D | | |
| +E,fra1/2 MS-Medium | | |
| MS-medium without 2-4-D, half concentrated | | |
| MS-macrosalts | | |
| KNO₃ | 19.0 | g/l |
| NH₄NO₃ | 16.5 | g/l |
| CaCl₂ × 2H₂O | 4.4 | g/l |
| MgSO₄ × 7H₂O | 3.7 | g/l |
| KH₂PO₄ | 1.7 | g/l |
| MS-microsalts | | |
| H₃BO₃ | 6.2 | g/l |
| MnSO₄ × H₂O | 16.8 | g/l |
| ZnSO₄ × 7H₂O | 10.6 | g/l |
| Na₂MoO₄ × 2H₂O | 0.25 | g/l |
| CoCl₂ × 6H₂O | 25.0 | mg/l |
| KJ | 0.83 | g/l |
| MS-vitamins | | |
| Glycine | 2.0 | g/l |
| Thiamine-HCl | 0.1 | g/l |
| Pyridoxine-HCl | 0.5 | g/l |
| Nicotinic acid | 0.5 | g/l |

The media were double concentrated sterile filtered. To prepare solid media 2% agarose is added in the same volume.

BASTA-Spray Solution

250 mg/l Basta (Hoechst, Frankfurt) and 0.% Tween 20

Production of Transgenic Plants

For the biolistic transformation of maize embryos, constructs for the over-expression of ZmMADS3 (sense) and for the suppression of ZmMADS3 expression (antisense) were prepared under the control of the actin promoter of rice (vector pAct1.cas). Only the ZmMADS3 gene specific 3′-region of the ZmMADS3 cDNA was used for the antisense transformation, however the complete cDNA was used for the transformation with the sense construct. Using the restriction enzymes EcoRI and SmaI the ZmMADS3 coding region of the sense construct can be cut out of genomic DNA (ca. 1300 bp). For the preparation of the antisense construct, cleavage sites for the restriction enzyme, HindIII and KpnI were introduced by PCR (see above). By means of an EcoRI and KpnI restriction digest, the cloned 3′-region of the ZmMADS3 is cut out with a circa 1 kb fragment of the actin promoter, so that the expected fragment has a length of approximately 1.3 kb. The herbicide resistance gene Pat (phosphinotrycin acetyl transferase) acts as a selection marker.

In four transformation experiments, a total of circa 350 embryos were bombarded with the ZmMADS3 antisense construct and circa 250 embryos were bombarded with the ZmMADS3 sense construct.

The transformation efficiency rates were approximately 0.8% for the ZmMADS3 sense transformation and 0.6% for the antisense transformation.

The plants were analysed in Northern and Southern blot analyses with regard to the integration of ZmMADS3 sense constructs or ZmMADS3 antisense constructs. The analysis of the plants with regard to the integration of the ZmMADS3 constructs was carried out with the rice actin promoter probe, which could detect full length integration of sense as well as antisense constructs.

Leaf material from putative transgenic plants of the regenerated plants (T0-generation) and the first to third progeny (T1- T3) were analysed for the integration of the transgene by means of Southern blots. 10 μg of each genomic DNA (isolated from leaf material) were digested overnight with the restriction enzymes Asp718 and XhoI, in accordance with the manufacturers' instructions, gel-electrophoretically separated, blotted and hybridised with the rice action promoter probe.

For Northern Blot analysis, RNA was isolated from leaves using TRIzol reagent (Gibco BRL) according to the manufacturers' instructions (in wild-type plants, ZmMADS3-transcripts are not found in leaves). The maceration of the tissues took place in a cooled swing-mill (Retsch) for 2-3 min. using steel beads. 10 μg total RNA each were electrophoretically separated on denaturating agarose gels, blotted overnight with 10×SSC on Hybond N⁺ membrane (Amersham) and fixed under 300 mJoule in a Stratalinker 1800 (Statagene). The filters were pre-hybridised for 5 hours at 65° C. using 100 μg/ml HS-DNA in CHURCH-Puffer (0.5 M NaH₂PO₄ (pH 7.2), 7% SDS, 1 mM EDTA). After over-night hybridisation using a probe concentration of 10⁶ cpm/ml the filters were washed a total of 6 times for 15 min. at 65° C. in decreasing SSC concentration (2×, 1×, 0.5× and 0.2×SSC, 0.1% SDS; 2×0.1×SSC, 0.1% SDS). The exposure of the filters on X-Omat AR films (Amersham) took place in trays with reinforced film at −70° C. The size of the RNA was determined using the Gibco BRL RNA-size standard 0.24-9.5 kb.

Antisense (AS) Plants: T0.4 and T0.11

A plant which had integrated both the marker gene and the antisense construct was regenerated from the experiments I and II(experiment I, T0.4 AS) and had a reduced seed set.

All 17 seed kernels germinated normally. Southern blot analysis showed that only 2 plants (T1.4.2AS and T1.4.3AS) contained two bands of the transgene each. One of the two bands represents the full length construct. An expression of the transgene was detected in all antisense plants carrying the antisense construct. Both plant were phenotypically normal with the exception that seed set on plant T1.4.3AS was reduced to about 50% in each row of the cob.

An integration of ZmMADS3 antisense construct could also be shown for the plant T0.11AS (experiment III). This plant, which contained a single integration of the antisense construct, was phenotypically characterised in that it developed two cobs. The male inflorescence corresponded to that of the wild-type plants.

Sense (S) Plants: T0.6 and T0.12 (See FIG. 6)

The integration of the ZmMADS3 sense construct was shown for two plants (T0.6S and T0.12S; FIG. 5; the arrows point towards the integration of the full length construct).

The T0.12 plants showed the most significant development disorders (FIG. 6a). They only achieved a height of about 30 cm and developed a hermaphroditic flower and no cobs at the apex. The pollination of the female flowers in apical inflorescence did not produce any growth of seeds and therefore no T1 generation could be analysed.

The T0.6S plants were small and developed an almost completely sterile tassel (FIG. 6b). The cobs were then pollinated with pollen from an A188 wild-type plant. A total of only 12 seed kernels developed, which did however germinate normally. Three plants of this T1-generation (T1.6.1S, T1.6.5S, T1.6.10S) died a few weeks after germination and could not be analysed. The remaining plants were examined in Northern and Southern Blot analyses. Two bands were detected in Southern Blot analysis which were not detectable in the A188 wild-type (WT) control. This double band indicates the integration of two constructs. One of the two cleavage sites must be deleted in one construct.

In Northern Blot analyses with total RNA from leaves, high ZmMADS3 transcript amounts in the T0 generation could be determined for the T0.6 plants.

Overview of the Off-Spring of the T0.6S Plants

Detection of DNA-fragments of the given sizes in Southern Blot analysis is indicated by “+”, failure to detect DNA fragments is indicated by “−”. Pheno-types that are deviant from the wild-type habitus are indicated by “PT”, or in cases of pronounced manifestation of the phenotype with “PT+”, wild-type habitus is indicated by “WT”. Information in brackets indicates phenotypes that were ambiguous, which could possibly result from environmental conditions.

| | | | | | | | | |
|---|---|---|---|---|---|---|---|---|
| Plant T1.6 | 2S | 3S | 4S | 6S | 7S | 8S | 9S | 11S |
| Transgene | + | − | + | + | + | + | − | + |
| Phenotype | | | | | | | | |
| Size | (PT) | (PT) | PT | PT+ | PT+ | WT | (PT) | (PT) |
| Tassel | PT | WT | PT | (PT) | PT | WT | WT | PT |
| Number of | 69 | 146 | 223 | 84 | 112 | 289 | 192 | 205 |
| Seeds | | | | | | | | |

In comparison to the control plants (T1.6.3S and T1.6.9S), the transgenic plants were characterised by a slightly smaller size, developmental disorders of the tassel and a slightly reduced number of seeds.

The tassel showed normal branching off habitus in comparison to the control plants, but developed almost 100% sterile flowers. This disorder was most pronounced in the T1.6.6S and T1.6.7S plants.

The cobs of the T1.6.2, T1.6.6 and T1.6.7 plants were characterised by a significantly reduced number of seeds in comparison to the wild-type plants.

Expression of the full length ZmMADS3 transgene was detected in all progeny plants containing an integration of the sense construct. The two bands were inherited as a single locus as can be seen in the T2 progeny of plant T0.6S (FIG. 5). All progeny plants missing the sense construct (7.4, 7.6, 7.9, 6.11. 6.13 and 6.14) were phenotypically normal (the tassel of plant 6.11 is also shown in FIG. 6c). On average, the size of the transgenic plants was 20% reduced, they contained only 9-10 nodes in comparison to 12-13 nodes in the WT plants, most cobs did not set seeds after pollination with pollen from A188 WT plants and male flower development was disturbed. The phenotype of male flowers of T2 and T3 progeny plants of T0.6 are shown in FIG. 6c-h: a completely sterile tassel developed at plants 7.3, 6.8 and 6.12 of the T2 generation and at most progeny plants of T2.6.6.6 (plant 6.6 in FIG. 5) in the T3 generation (FIG. 6c). All side branches were sterile at plants 7.1, 7.2, 7.10, 7.12, 6.2 and 6.6, whereas the main branch was normal (FIG. 6c). Male flowers of transgenic plants (FIG. 6e and f) were less developed than WT flowers (FIG. 6d). Longitudinal sections in regions indicated by boxes in FIG. 6d and f showed that male flower organs were transformed into leaf like structures (see arrows in FIG. 6h) in the plants containing an integration of the sense construct. FIG. 6g shows a comparable section of a WT plant.

In summary it can be ascertained that the results give the indication that plants transformed with a ZmMADS3 sense construct demonstrate growth disorders and disorders in the development of flowers (male and female). Thus the organs and tissues are affected, which showed ZmMADS3 expression in wild-type plants. This indicates that this is a genetic effect. The effect may either be due to over-expression of ZmMADS3 or due to co-suppression.

### REFERENCES CITED

An et al. (1985) EMBO J. 4, 277-287

Becker et al. (1994) Plant J. 5, 299-307

Brettschneider et al. (1997) Theor. Appl. Genet. 94, 737-748

Bytebier et al. (1987) Proc. Natl. Acad. Sci. USA 84, 5345-5349

Canas et al. (1993) Plant J. 6, 597-604

Chan et al. (1993) Plant Mol. Biol. 22, 491-506

Coen and Meyerowitz (1991) Nature 353, 31-37

Dellaporta et al. (1983) Plant Mol. Biol. Rep. 1, 19-21

Dresselhaus et al. (1994) Plant J. 5, 605-610

Dresselhaus et al. (1996a) Plant Mol. Biol. 31, 23-34

Dresselhaus et al. (1996b) Plant Mol. Biol. 30, 1021-1033

Gould et al. (1991) Plant Physiol. 95, 426-434

Green and Phillips (1975) Crop Sci. 15, 417-421

Hiei et al. (1994) Plant J. 6, 271-282

Hoekema (1985) The Binary Plant Vector System, Off-setdrukkerij Kanters B. V., Alblasserdam, Chap. V

Holsters et al. (1978) Mol. Gen. Genet. 163, 181-187

Ishida et al. (1996) Nature Biotechnology 14, 745-750

Jähne et al. (1995) Euphytica 85, 35-44

Kater et al. (1998) Plant Cell 10, 171-182

Koltunow et al. (1995) Plant Physiol. 108, 1345-1352

Lit et al., Plant Mol. Biol. 20 (1992), 1037-1048

Logemann et al. (1987) Anal. Biochem. 163, 16-20

Luo et al. (1999) Proc. Natl. Acad. Sci. USA 96, 296-301

Maheshwari et al. (1995) Critical Reviews in Plant Science 14 (2) 149-178

Mandel et al. (1992a) Cell 71, 133-143

Mandel et al. (1992b) Nature 360, 273-277

Matzk et al. (1995) Sex. Plant Reprod. 8, 266-272

Matzk et al. (1997) Hereditas 126, 219-224

Mena et al. (1995) Plant J., 8, 845-854

Mena et al. (1996) Science 274, 1537-1540

Michaelis and Amasino (1999) Plant Cell 11, 949-956

Mooney et al. (1991) Plant Cell Tiss. & Org. Cult. 25 209-218

Murashige and Skoog (1962) Physiol. Plant 15, 473-497

Nordhorst and Lörz (1993) J. Plant Physiol. 142, 485-492

Ohad et al. (1999) Plant Cell 11, 407-415

Potrykus (1990) Physiol. Plant, 296-273

Raineri et al. (1990) Bio/Technology 8, 33-38

Richmann and Meyerowitz (1997) Biol. Chem. 378, 1079-1101

Richert et al. (1996) Plant Sci. 114, 93-99

Rotino et al. (1997) Nat. Biotechnol. 15, 1398-1401

Sambrook et al. (1989) Molecular Cloning, A Laboratory Manual, 2^{nd}. Edition, Cold Spring Harbor Press, NY,

Sheldon et al. (1999) Plant Cell 11, 445-458

Stirn et al. (1995) Plant Sci. 106, 195-206

Vielle-Calzada et al. (1996) Science 274, 1322-1323

What is claimed is

1. A nucleic acid molecule for use in cloning and expressing in a plant the nucleic acid sequence encoding a protein influencing flower structure, function and/or its seed and/or fruit development which is selected from the group consisting of
(a) the nucleic acid sequence defined in SEQ ID No. 1, or complementary strand thereof,
(b) a nucleic acid sequence encoding a protein or peptide with the amino acid sequence defined in SEQ ID No. 2, or complementary strand thereof,
(c) a nucleic acid sequence which hybridizes to the nucleic acid sequence defined a) or b), or a complementary stand thereof, which has a degree of identity of more than 70%, and
(d) a nucleic acid sequence which is degenerate, as a result of the genetic code, to the nucleic acid sequence defined in a), b), c), or a complementary strand thereof.

2. The nucleic acid molecule of claim 1, which is derived from maize.

3. The nucleic molecule of claim 1 which is a DNA or cDNA, or RNA molecule.

4. A vector comprising the nucleic acid molecule of claim 1.

5. The vector of claim 4, which is a bacterial or viral vector.

6. The vector of claim 4, wherein the nucleic acid molecule is operably linked to at least one regulatory element, in particular in an antisense or sense orientation.

7. The vector of claim 6, wherein the regulatory element functions at either the 5′ or 3′ end of said nucleic acid.

8. The vector of claim 7, wherein the 5′ regulatory element is a promoter, in particular the CaMV 35S promoter or the actin promoter.

9. The vector of claim 7, wherein the 3′ regulatory element is a termination and poly A addition sequence, in particular from the NOS gene of Agrobacterium tumefaciens.

10. The vector according to claim 4, which furthermore contains T-DNA, in particular either the left, or the right, or both left and right T-DNA borders.

11. The vector according to claim 10, wherein the nucleic acid molecule, optionally in conjunction with at least one regulatory element, is located within the T-DNA or adjacent to it.

12. A transgenic host cell containing the vector of claim 4 or a cell deriving therefrom.

13. The host cell of claim 12, which is a plant, yeast or bacterial cell, in particular a cell from a monocotyledonous or dicotyledonous plant or a cell deriving therefrom.

14. A cell culture, preferably a plant cell culture comprising a cell according to claim 12.

15. A method of genetically modifying a cell by transforming a cell with a nucleic acid molecule of claim 1 or a vector according to claim 4 wherein the nucleic acid molecule is expressible in the cell.

16. The method of claim 15, wherein the cell is a plant, bacterial or yeast cell.

17. The method of claim 15, wherein the transformed cell is regenerated into a differentiated plant.

18. The method of claim 15 wherein the cell is transformed by transfer of the nucleic acid molecule or vector from a bacterium to the cell.

19. The method of claims 15, wherein the cell is transformed by direct uptake of nucleic acid sequences, or by microinjection of nucleic acid sequences, or particle bombardment.

20. A method for isolating node, flower and embryo sac genes from a plant, whereby a nucleic acid sequence of claim 1 is used to screen nucleic acid sequences derived from the plant.

21. A plant comprising a host cell according to claim 12 or produced according to a method according to claim 15.

22. Propagation and harvest material, in particular seeds and plant tissue, comprising a host cell according to 12.

23. A method for the production of a genetically modified plant with a modified flower, seed and/or fruit structure, or modified function or development, wherein a plant cell is transformed with a nucleic acid molecule according to claim 1 or a vector according to claim 4 and the transformed cell is regenerated into a plant.
